(19) United States
(12) Patent Application Publication
(10) Pub. No.: US 2005/0026200 A1
(43) Pub. Date: Feb. 3, 2005

(54) Method for error detection and increased confidence of sample decoding

(76) Inventor: David P. Holden, Burlingame, CA (US)

Correspondence Address: HARNESS, DICKEY & PIERCE, P.L.C., P.O. BOX 828, BLOOMFIELD HILLS, MI, 48303, US

(21) Appl. No.: 10/901,474

(22) Filed: Jul. 28, 2004

Related U.S. Application Data
(60) Provisional application No. 60/490,561, filed on Jul. 28, 2003.

(51) Int. Cl. C12Q 1/68; G06F 19/00; G01N 33/48; G01N 33/50

(52) U.S. Cl. 435/6; 702/20

(57) ABSTRACT

A system and method for providing an error correction or determination for a data set or string regarding an image or detection of a selected element or component at an interrogation site. The interrogation site can include a bead or other appropriate detection system. The detection system and generated data string includes an error correction or detection indication that ensures that the data transferred to the analysis system is substantially error free.

### CROSS-REFERENCE TO RELATED APPLICATIONS

This application claims the benefit of U.S. Provisional Application No. 60/490,561, filed on Jul. 28, 2003. The disclosure of the above application is incorporated herein by reference.

### FIELD

The present disclosure and claims relate generally to a system for error detection and correction; and more specifically to a system for detecting an incorrectly read or transmitted data stream.

### INTRODUCTION

Various analyses can be performed by collecting information regarding a certain event. The information can be decoded and digitized for processing. Processors of various kinds can be provided to both decode and performs selected steps of an analysis.

### DRAWINGS

The skilled artisan will understand that the drawings, as described below, are for illustration only. The drawings are not intended to limit the scope of the present teachings in any way.

FIG. 1 is a detail view of an interrogation site according to an embodiment;

FIG. 2 is a flow chart of an error detection system according to an embodiment; and

FIG. 3 is a schematic view of a transmission system according to an embodiment.

### DESCRIPTION OF VARIOUS EMBODIMENTS

The following teachings of various embodiments is merely exemplary in nature and is in no way intended to limit the teachings, its application, or uses.

A system can be provided to decode a selected portion or component, such as a selected genetic sequence. For example, a bead can be used to analyze various biological components, such as certain DNA or base pair segment fragments. These beads can be positioned in selected arrays, such as the BEADARRAY™ sold by Illumina of San Diego, Calif. The beads are loaded with any appropriate mechanism and/or according to any appropriate method such that a selected component adheres to the bead if the selected component is present within the sample to be analyzed.

Briefly, the bead can include a portion to provide an indication. For example, the bead may be excited and reflect a selected wavelength of light. The bead, however, may respond or change its indication due to the presence of the selected sample that interconnects or interacts with the bead. The beads can respond by producing a different color when excited by a selected excitation beam. The beads can also include a particular resonance frequency which can be detected. Nevertheless, the bead can be analyzed for the selected change that can depend upon the presence of a selected component.

With reference to Table 1, below, an exemplary system includes beads that can include two colors, for example color 1 and color 2, wherein a zero can stand for red and a one stand for green. Therefore, as illustrated in Table 1 below, four beads can be decoded and include two bits, exemplary referred to as sample bits, of information to identify each type of bead. A two-stage decode process for each of the four bead types is illustrated.

TABLE 1
Sample Decoded Bead Data

| Bead | Bit 1 | Bit 2 | Color 1 | Color 2 |
|---|---|---|---|---|
| A | 0 | 0 | r | r |
| B | 0 | 1 | r | g |
| C | 1 | 0 | g | r |
| D | 1 | 1 | g | g |

Each decoded bead creates or includes two bits, one for each of the colors that the bead can include. The data defines a characteristic indication for that bead or interrogation site. Four beads are illustrated, A, B, C, and D for the different types of beads that can be decoded. Nevertheless, each bead includes a two-stage decode wherein any code or bit produced is determined to be valid. That is, once the bit for the particular color is determined or decoded that bit is determined to be valid and is never checked to ensure that the bit is transmitted correctly. Therefore, any classification of the beads is at least slightly unknown due to the fact that the only information that is transmitted is determined to be valid, but can actually include invalid information.

An error correction indication, however, can be added to the data or process to increase the confidence of the information being transmitted. For example, the error correction indication can include various error correction bits or bytes that can be added to the process. Error correction bits or bytes can includes parity bits, checksums, or other appropriate error correction bits that can be included in the data transmission to ensure that the data that is transmitted is correct data. It will be understood that the error detection or correction indication can include a single or plurality of bits.

Including an error correction bit can increase the confidence in the data that is transmitted because an additional level of checking has occurred to ensure that the data is correct. Also the data is checked again at the processor or storage area. Therefore, the data can be checked both at its creation and prior to analysis to ensure substantially valid data. Although an error can still occur, the data is less likely to be invalid after the error detection bit has been checked because of the additional step or process of creating the error correction bit.

For example, a parity bit can be calculated by summing the bits in a pre-specified number of bit modulus. With reference to Table 2 below, a parity bit, also referred to as an error detection bit, for each of the beads is illustrated. The parity bit can be provided to ensure that the sum of all non-zero bits is either even or odd. Here the parity bit is provided to ensure an even parity.

TABLE 2
Sample Decoded Bead Data with Error Correction Code

| | | | Even Parity | | |
|---|---|---|---|---|---|
| Bead | Bit 1 | Bit 2 | Bit | Color 1 | Color 2 |
| A | 0 | 0 | 0 | r | r |
| B | 0 | 1 | 1 | r | g |
| C | 1 | 0 | 1 | g | r |
| D | 1 | 1 | 0 | g | g |

In this example, the even parity bits are 0, 1, 1, 0 for each of the beads A, B, C, and D, respectively. The parity bit provides a method of ensuring that the bits being transmitted for a particular bead are correct. The parity bit transforms the bits for the particular bit to ensure that an even parity exists. If the data received by the processor does not have an even parity, an even sum for the non-zero bits, then the data for that bead can be discarded. Thus, the validity of the data for the beads is increased.

Therefore, the information that is transmitted to the processor includes a higher level of validity and/or confidence than a system that did not include such an error detection bit. The additional process of adding the parity bit provides an additional step that can be performed correctly to ensure that the data is valid. If the parity is not correct then the data for the bead can be discarded such that the data does not corrupt the entire data set. Including the parity bit may not ensure an absolutely error free data set, but greatly decreases the probability that an error in the data set during transmission has occurred.

Other appropriate error detection methods can also be used or included to further ensure valid data transmission. A higher confidence can be maintained by the use of a CRC where several bits are produced for each bead. The greater the number of bits used to determine validity the greater the chance to detect an error. Alternatively, other known or newly developed checksums can be used to validate the data being created regarding the various beads depending upon the desired results. In this case the data could be summed according to a selected formula to ensure that a selected value is achieved to ensure that all of the data is transmitted properly.

The data set can be created according to various and appropriate systems. For example, the beads can be interrogated in a flow chamber. The interrogation can be any appropriate interrogation, such as excitation with a light source. In a flow chamber, the beads can pass an interrogation point and can be decoded into a plurality or string of bits. The bits relate to the decoding of the bead, such as the color produced by the bead during the interrogation. Alternatively, as discussed above the data can relate to a bead or plurality of beads positioned and interrogated in an array, such as the BEADARRAY™. Regardless the system that is decoding the various beads, the decoded beads, as data bits, is transmitted to a processor, that may also be referred to as a decoder, for analysis and comparison.

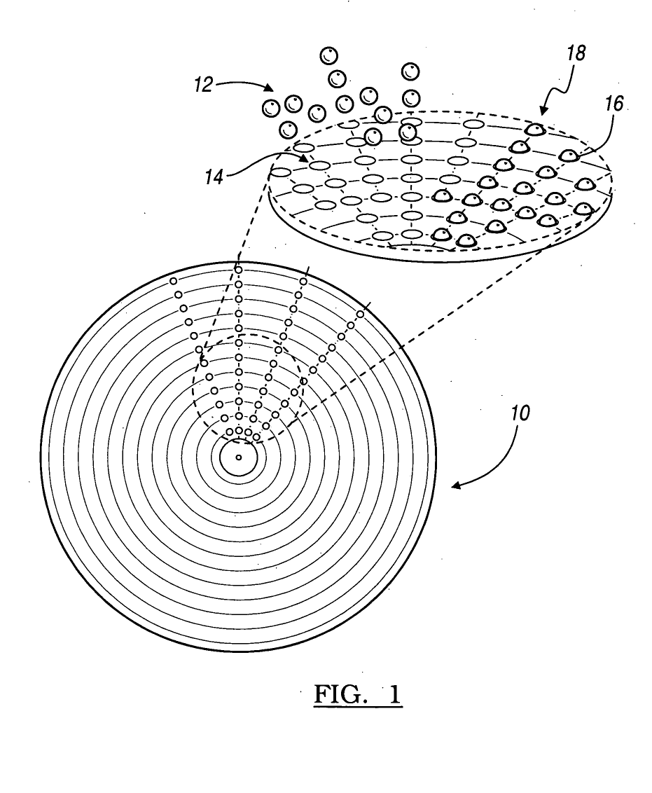

The following teachings relate to an exemplary method and system that can be used to detect errors for transmission of data relating to decoding beads. With reference to FIG. 1, an array system 10 is illustrated. In the array system 10, a bead 12 is positioned relative to an array 14. The array 14 can be any appropriate array, but can include a plurality of fibers, such as optic fibers, that includes a well 16 to contain the bead 12. A plurality of the beads 12 can be positioned in a plurality of the wells 16 to form a loaded array 18.

The beads 12 are generally optically encoded or can be encoded in libraries for detection of comparison to sample beads. In addition, the beads 12 generally contain a site which can interconnect with a selected component, such as a selected portion of a gene, to alter or induce a response to a selected excitation mechanism, such as a laser. Generally, the beads can be optically active such that when excited by a selected radiation they will emit a selected wavelength of light depending upon whether a selected component or species is interconnected with the bead 12. Therefore, either before or once the beads 12 are positioned in the well 16, to form the loaded array 18, a chemical species is allowed to interact with the beads 12.

Once the species is allowed to interact with the beads 12 and allowed to interconnect with the bead 12, the loaded array 18 is also a loaded sample array. The beads 12 can then be detected in a selected detection system, such as the Sherlock™ 1000 Array Scanner, produced by lllumina™ of San Diego, Calif. The array scanner can illuminate a sequential or simultaneous plurality of the beads 12 positioned in the loaded array 18 to produce a signal from each of the beads 12. The signal produced can be transformed or decoded into data with any appropriate processor, that may also be referred to as a decoder. The data may be arranged in a table format, such as the data set illustrated in Table 1, for each of the bead locations. The array scanner is able to digitize the data depending upon the selected information and can transfer the information to an analysis system.

The decoding of the beads 12, as discussed above, produces the plurality of bits for each of the beads 12 interrogated. Therefore, to ensure a proper validation of the data that is being transmitted depending upon the decoding of the beads, the error correction bit or bytes can be added to the data being transmitted. This occurs substantially immediately after the beads 12 are decoded to ensure that any further transmission of the data is substantially error free or errors in the data transmitted can be determined.

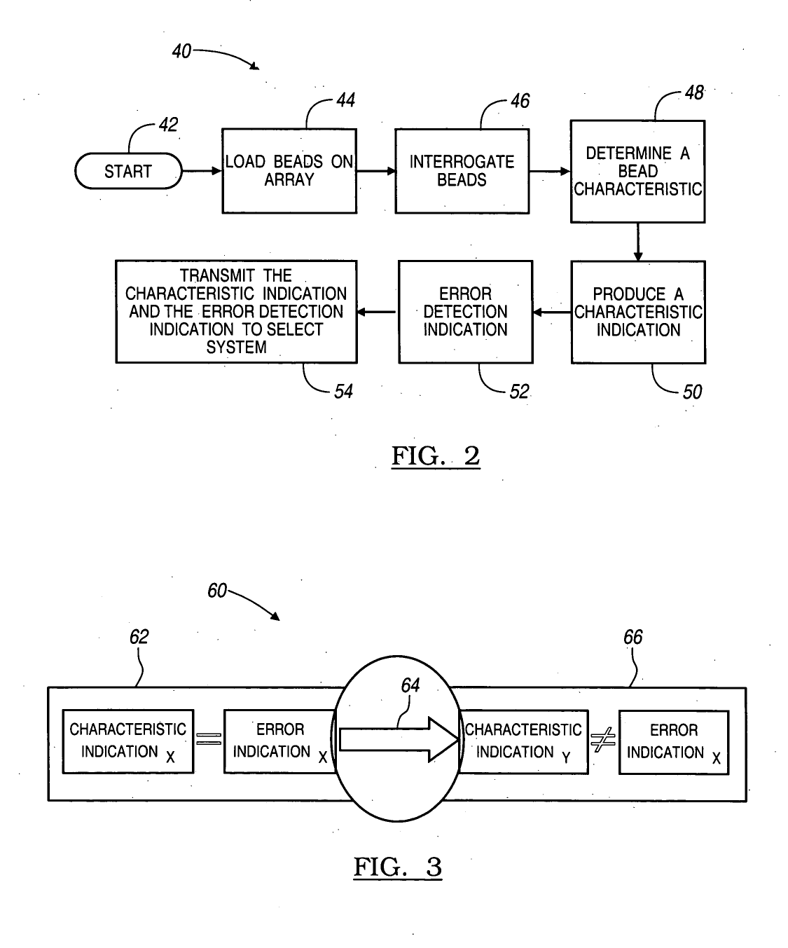

Generally, the decoding and transmission of the bead data can follow a decoding and error detection method 40, with reference to FIG. 2. The method generally starts at block 42 after which beads are loaded into an array or loaded for other appropriate interrogation, in block 44. It will be understood that beads are merely exemplary and not limiting. Simply, an interrogation site exists that will be interrogated to produce a sample indication. To this sample indication, that can include a bit or plurality of bits, an error correction indication can be added. The beads 12 loaded into the array can either include the optical tags for a sample to be added later or can be loaded with the components to be interrogated already incorporated. The beads are then interrogated, in block 46, to produce a selected excitation if the component has interconnected with the beads.

With reference to the BEADARRAY™, the array is interrogated using light, such as a laser beam, in an array scanner. Nevertheless, it will be understood that the beads can be interrogated in a plurality of ways, depending upon the particular array or system. For example the error detection method can be used in conjunction with a flow bead system where the beads are not stationary, but the beads are mobile relative to the scanner.

Nevertheless, after the beads are interrogated, the beads are decoded to produce a data set, such as a plurality of bits, to define the bead such as including a selected component or not including a selected component depending upon the response of the bead to the interrogation. After decoding the beads, a data set is produced, in block 50, after which error detection is added, in block 52, to the data set produced in block 50. After the error detection is added, the data including the error detection portion can be sent or transmitted to a selected system, in block 54, such as an analysis system or database. The data set including the error detection portion can be submitted substantially increasing its validity after being included in a following analysis or database.

As discussed above, the error detection added in block 52 can be any appropriate or selected error detection indication. Exemplary error correction indications include parity bits, check sums, CRCs. The main difference being the amount of confidence given to the data transmitted depending upon the amount of error detection data provided to the data transmission stream. It will also be understood that the present method can be used with any appropriate detection system. For example, the detection system can be used to decode and transmit data for a plurality of beads positioned in the BEADARRAY™. Nevertheless, the data can also be produced by decoding a plurality of beads which are moved past an interrogation area or window to also produce a data stream or set. Therefore, the data being produced is simply illustrative of any appropriate information that can be provided by detecting a plurality or a selected component on a plurality of beads or sample wells.

With reference to FIG. 3, a diagrammatic view of a transmission system 60 is illustrated. A first or scanner system 62 interrogates the selected interrogation or sample site. A characteristic indications is produced in the first system. Also, in the first system an error indications is added to the characteristic indications. Exemplary the error indicationₓ is equivalent to the characteristic indicationₓ. The error indications can be any appropriate indicationₓ such as a parity bit, parity byte, checksum, etc.

Regardless of the error indicationₓ chosen, it is transferred with the characteristic indicationₓ along line 64 to a second system 66, such as a workstation, database, main frame or any other appropriate system. During the transmission an error can be introduced into the characteristic indications to transform or change it to an invalid characteristic indications. The error can occur because of interference or communication errors. Nevertheless, in the second system the characteristic indications is no longer equivalent to the error indications that is also sent along line 64. Thus the second system 66 is able to determine that the characteristic indications is not valid and can be ignored, if desired. This greatly increases the confidence in the transmitted indication when the error indication is also transmitted with the characteristic indication.

Although the above is related particularly to the detection and decoding of various biological components on the beads or on an array, it will be understood that the present system can be used on any appropriate detection or decoding system. For example, the information being provided from the sample detection or interrogating system can interrogate the presence of selected chemicals, biological components, or other selected materials. Moreover, the beads can include properties other than colors or various energy emissions that are being detected. Again, the bead can be any appropriate interrogation site and a bead is merely exemplary. Other energy emissions, such as ultraviolet or infrared can also be detected and other features such as size, mass, or resonance can also be detected. Nevertheless, the information is decoded into a series of bits which is transmitted to a processor with the error detection bits. It is the processor that uses the error detection bit to ensure that the information that is transmitted is substantially valid.

The description of the teachings is merely exemplary in nature and, thus, variations that do not depart from the gist of the teachings are intended to be within the scope of the teachings. Such variations are not to be regarded as a departure from the spirit and scope of the teachings.

1. A method of increasing the validity of data transmitted from a detection system, comprising:
evoking a selected response depending upon whether the selected biological component is present;
decoding the selected response into a sample indication;
adding an error correction indication to said sample indication; and
transmitting the sample indication and the error detection indication to a selected analysis system.

2. The method of claim 1, wherein said error correction indication is selected from at least one of an even parity bit, an odd parity bit, a parity byte, a checksum, a cyclic-redundancy-check, or combinations thereof.

3. The method of claim 1, wherein said error correction indication is added to said sample indication prior to said transmitting said sample indication.

4. The method of claim 1, further comprising:
positioning a biological component at an interrogation site to interact with said site when said biological component is present; and
interrogating the interrogation site to evoke the selected response.

5. The method of claim 4, wherein decoding the selected response into a sample indication, includes:
detecting a response of the interrogation site due to the interrogation; and
producing said sample indication due to the detection.

6. The method of claim 5, wherein said response includes a size, a mass, a resonance, an energy emission, a frequency, an infrared energy, and combinations thereof.

7. The method of claim 1, further comprising:
reading said transmitted sample indication and error detection indication; and
determining that the sample indication is at least one of valid or invalid due to the error detection indication.

8. The method of claim 4, wherein positioning a biological component at an interrogation site includes at least one of positing the biological component in a well, a bead, a bead disposed in a well, an array, or combinations thereof.

9. A system to substantially confirm validity of a data transmission, the system comprising:
a scanner positioned relative to an interrogation site;
a sample positioned relative to said interrogation site;
a decoder to determine a selected characteristic of said sample and to produce a characteristic indication to define said selected characteristic; and
an analyzer to analyze said characteristic indication produced by said decoder;
wherein said decoder adds an error correction indication to said characteristic indication to increase a confidence in the transmission success of said characteristic indication.

10. The system of claim 9, wherein said sample includes:
a bead including said selected characteristic that is created or changed when a component of interest interconnects with said bead.

11. The system of claim 10, wherein said scanner interrogates said bead.

12. The system of claim 10, wherein said characteristic includes an energy emission, a size, a mass, an infrared emission, a resonance, a frequency, and combinations thereof.

13. The system of claim 9, wherein said error correction indication includes at least one of a parity bit, a parity byte, a checksum, a cyclic-redundancy-check, or combinations thereof.

14. The system of claim 9, wherein said sample includes at least a portion of a gene.

15. The system of claim 9, wherein said interrogation site includes a bead, an array, a well, or combinations thereof.

16. A method confirming a validity of a sample indication, comprising:
determining a sample indication by interrogating an interrogation site in a first system;
forming a digitized sample indication to be transferred to a second system;
adding an error detection indication to said digitized sample indication in said first system; and
confirming said digitized sample indication in said second system with said error detection indication.

17. The method of claim 16, wherein said interrogation site includes at least one of a bead, a well, an array, a bead in a well, and combinations thereof.

18. The method of claim 16, wherein said sample includes at least one of a chemical species, a portion of a gene, a cell, and combinations thereof.

19. The method of claim 16, wherein determining a sample indication by interrogating an interrogation site in a first system, includes:
exciting said interrogation site; and
detecting at least one of an energy emission, a light emission, an infrared emission, a resonance, a frequency, a size, a mass from said sample.

20. The method of claim 16, wherein adding an error detection indication, includes adding at least one of a parity bit, a parity byte, a checksum, a cyclic-redundancy-check, or combinations thereof.
